(19) United States
(12) Patent Application Publication
(10) Pub. No.: US 2002/0127741 A1
(43) Pub. Date: Sep. 12, 2002

(54) Free Analyte detection system

(76) Inventors: Miquel Sales Amill, Barcelona (ES); Jose Manuel Delgado Calvo Flores, Granollers (ES); Josep Serra, Barcelona (ES); Bjorn Dahlback, Malmo (SE)

Correspondence Address: TESTA, HURWITZ & THIBEAULT, LLP, HIGH STREET TOWER, 125 HIGH STREET, BOSTON, MA, 02110, US

(21) Appl. No.: 10/022,481

(22) Filed: Dec. 18, 2001

Related U.S. Application Data
(60) Provisional application No. 60/256,600, filed on Dec. 19, 2000.

(51) Int. Cl. G01N 33/546

(52) U.S. Cl. 436/533

(57) ABSTRACT

Particle-based methods, compositions, and kits are provided for the detection of the free form of a member of a binding pair which may be found free or in a bound state in a sample. Inhibition and direct assay formats are provided. In one embodiment, the invention provides diagnostic tools for the diagnosis of thrombophilia through the detection of free protein S.

### CROSS-REFERENCE TO RELATED APPLICATIONS

This application is based on and claims priority to provisional application Serial No. 60/256,600 which was filed on Dec. 19, 2000.

### FIELD OF THE INVENTION

The invention relates to particle-based assays, compositions, and kits for qualitative and quantitative detection of the unbound form of a member of a binding pair. In one aspect, the invention relates to particle-based assays, compositions, and kits used to diagnose thrombophilia based on the detection of free protein S in plasma samples.

### BACKGROUND OF THE INVENTION

Changes in the concentrations of molecular binding partners (e.g., receptors and their ligands) can result in pathological conditions manifested as changes in the ratio of free to bound members of a binding pair. By distinguishing between the bound member of a binding pair and its unbound form, such changes can be monitored to provide a diagnostic assay. Methods which distinguish between free and unbound forms of a binding pair generally require the separation of the bound member of a binding pair from its free form. Besides adding extra steps to any diagnostic assay, this requirement reduces the sensitivity of the assay due to potential loss of the member of the binding pair during the separation step.

An agglutination reaction involves the in vitro aggregation of microscopic carrier particles onto which a member of a binding pair (e.g., an antibody or an antigen) has been immobilized. When an antibody is the member of the binding pair attached to the particle, binding of a specific antigen to the antibody will only trigger agglutination if the epitope recognized by the antibody is repeated at least twice (i.e., forming a particle:antigen:particle complex). Agglutination may be directly detected and quantitated either visually or through the use of devices which measure light scatter. Alternatively, the amount of non-agglutinated particles may be determined to provide an inhibition measure of the number of agglutinated particles. In an inhibition assay format, an analyte, e.g., an antibody or antigen, is detected by determining the extent to which the antigen or antibody being assayed will interfere with agglutination. See, e.g., U.S. Pat. No. 4,184,849.

Agglutination assays known in the art offer the advantages of allowing the sensitive detection of small amounts of analyte in a low cost assay format without the use of radioactivity. However, many of these assays still suffer the disadvantages discussed above of not being able to distinguishing the presence of the bound form of a member of a binding pair from the unbound form without a separation step or a precipitation step.

Thrombotic diseases are examples of pathological conditions in which diagnosis of the disease would benefit from the ability to distinguish between the free form of a member of a binding pair and the bound form of a member of a binding pair in a biological sample. Thrombotic diseases occur when the hemostatic balance between pro- and anticoagulation forces is shifted in favor of coagulation. Although many factors can lead to a hypercoagulable state, protein S, a 69 kd glycoprotein, plays an integral part in this pathway by acting as a cofactor of activated protein-C (APC) allowing it to degrade coagulation factors Va and VIIIa. Protein S is found in the blood of healthy individuals in two forms, a free form responsible for its anticoagulant activity, and a non-active form in which it is bound to C4b binding protein (C4BP), a member of the complement pathway.

In the blood of healthy individuals, 40% (˜10 μg/ml) of protein S is present in its free active form while 60% (˜15 μg/ml) is found complexed in a nonactive form with C4BP. Low levels of free protein S (and therefore lower activity of APC, and high levels of coagulating factors, Va and VIIIa) are associated with hypercoagulable states and a risk for thrombotic diseases. See, e.g., Faioni, et al., Thromb. Haemost. 78: 1343-1346 (1997); Simmonds, et al., Blood 89: 4364-4370 (1997); Comp, et al., Blood 67: 504-508 (1986); Sallah, et al., Thromb. and Haemost. 73: 1259 (1997).

Protein S has been assayed using both functional and antibody-based assays. Functional assays rely on quantitating the clotting activity of a sample or by measuring the photometric signal produced by a chromogenic substrate reactive with clotting factors downstream of protein S. See, e.g., U.S. Pat. No. 5,439,802 and U.S. Pat. No. 5,308,756. Because these assays do not measure protein S directly, requiring the functional interactions of many other components of the clotting cascade, they lack the sensitivity and specificity necessary for a clinical diagnostic assay.

Antibody-based assays for the detection of free protein S have been developed using monoclonal antibodies which react specifically with protein S. See, e.g., Murdock, et al., Clin. Lab. Haematol. 19(2): 111-4 (1997); Amiral, et al., Blood Coagulation and Fibrinolysis 5: 179-186 (1994); Bertina, et al., Thromb. Haemostas., 53(2): 268-272 (1985). Heterogenous immunoassays (e.g., enzyme-linked immunosorbent assays (ELISAs), enzyme-linked sorbent assays (ELSAs), and rocket immunoelectrophoresis methods) have also been used, but require the separation of bound protein S from free protein S either during the preanalytical step, by precipitation of bound protein S with polyethyleneglycol, or during the development of the immunoassay, by washing away the complex that does not bind to the solid phase (see, e.g., Giri, et al. (1988), Thromb Haemost 79: 767-772). This article describes a main improvement over previous microtiter-based enzyme immunoassays (EIA) which required the development of highly specific monoclonal antibodies against the binding site of protein S to its natural ligand C4BP. In this method, purified C4BP attached to the solid phase captures free protein S. The method, although advantageous, is time consuming, difficult, and expensive to automate.

Because the detection of free-protein S provides one of the most valuable parameters for the clinical diagnosis of thrombotic disease, it is desirable to provide a rapid, low-cost, analyte detection method able to detect and quantitate the amount of free protein S in a sample which does not require separating bound from unbound forms of protein S. Such an assay would have general utility in detecting altered binding concentrations between molecular binding partners implicated in pathological conditions.

### SUMMARY OF THE INVENTION

The present invention addresses the need for an analyte detection assay which enables the qualitative and quantitative detection of the free form of an analyte which can exist free in solution as well as bound to another molecule. The compositions, kits, and methods described herein provide tools for clinical diagnostic assays which are low cost, require minimal steps, and require no radioactivity. In one embodiment of the invention, compositions, kits, and assays of the invention can be used to diagnose thrombophilia based on the detection of free protein S in plasma samples.

In a first aspect according to the invention, a method is provided for direct detection of an unbound form of a first member of a binding pair which comprises a first and second member, each member bindable to the other through one and only one binding site. A first particle bound to the second member is reacted with a sample of interest. Any unbound first member in the sample binds to the second member bound to the first particle, forming a first complex comprising first member:second member:first particle which does not produce a significant change in turbidity in the sample. A second particle bound to a third member which is different from the second member and capable of binding to the first member is then provided. This forms a second complex between the third member bound to the second particle and the first complex (e.g., a second particle:third member:first member:second member:first particle complex) which is detectable as a significant increase in the turbidity of the sample and may be quantitated, for example, by measuring the change in turbidity of the sample over time. In performing the assay, the first particle bound to the second member and the second particle bound to the third member may be added sequentially or simultaneously. By providing a second member which is C4BP, the method can be used to detect the presence of free protein S. By using a first particle bound to a binding partner of the first member and which has only a single binding site for that first member, the method is highly specific for only the free form of the first member.

In a second aspect of the invention, an inhibition assay format is provided for detecting the unbound form of a first member of a binding pair. In this embodiment, a first particle bound to second member is again reacted with a sample to form a first complex with any unbound first member in the sample. A second particle, this time bound to the first member, is then provided which binds to any first particle bound to second member which is not already bound to the first member that was in the sample. The second complex formed by this reaction (i.e., second particle:first member:second member:first particle) is detected, for example, by measuring turbidity, providing a measure which is inversely proportional to the amount of unbound first particle that was in the sample.

In another aspect of the invention, compositions and kits are provided to perform the assays. Compositions used to perform direct assays comprise a first particle bound to the second member, and a second particle bound to the third member. Compositions used to perform inhibition assays comprise a first particle bound to the second member and a second particle bound to the first member. Compositions may be pre-packaged in a kit with appropriate buffers and controls, and may optionally include a set of printed instructions. In preferred embodiments of the invention, the second member which is bound to the first particle is C4BP, and the compositions and kits provide reagents for the detection of free protein S.

The foregoing and other objects, aspects, features, and advantages of the invention will become apparent from the following description and from the claims.

### BRIEF DESCRIPTION OF THE DRAWINGS

The objects and features of the invention can be better understood with reference to the following detailed description and accompanying drawings, in which like reference characters generally refer to the same parts throughout the different views.

FIGS. 1A-1C is a schematic diagram of the steps in a direct assay format according to one embodiment of the invention.

FIGS. 2A-2C is a schematic diagram of the steps in an inhibition assay format according to one embodiment of the invention.

FIG. 3 is a graph showing measurement of free protein S in plasma samples using the direct assay format shown schematically in FIG. 1. The graph shows the change in absorbance (vertical axis) over time (horizontal axis) in a sample from a healthy individual (squares) and in a sample from a patient with thrombophilia (diamonds).

FIG. 4 is a graph showing measurement of free protein S in plasma samples using the direct assay format shown schematically in FIG. 1. The graph shows a calibration curve in which change in absorbance (vertical axis) is plotted against known concentrations of free protein S (horizontal axis; 100%=1 IU/ml) measured using the direct assay format.

### DETAILED DESCRIPTION OF THE INVENTION

The present invention provides methods, compositions, and kits for the detection of the free form of a member of a binding pair which may be found free or in a bound state in a sample. The method provides advantages over methods known in the art in not requiring the separation of free and bound forms of the molecule prior to detection, thus providing a robust assay format which has the sensitivity and reproducibility necessary for a diagnostic test. By providing a first particle bound to the natural binding partner of a first member of a binding pair and which has a single binding site for the first member, the assay is highly specific for the free form of the first member of a binding pair. In a preferred embodiment, a method is provided for the diagnosis of a thrombotic disease through the detection and/or quantification of free protein S.

The present invention provides both direct and inhibition assay formats, although many permutations of these formats will be obvious to those of skill in the art and are encompassed within the scope of the invention.

### Definitions

In order to more clearly and concisely describe and point out the subject matter of the claimed invention, the following definitions are provided for specific terms which are used in the following written description and the appended claims.

As used herein, the term “binding pair” means a complex comprising at least first and second members which have reversible specific binding affinities for each other and which exist in both free and bound forms in solution. In a preferred embodiment of the invention, a binding pair has an affinity constant greater than 10-7 moles/liter.

The term “first member” means an analyte, i.e., a molecule which is to be detected. The term “analyte” is used herein interchangeably with “first member,” and refers to any substance for which a binding partner exists or can be generated for, and can be naturally occurring in a biological sample, or artificially created.

The “second member” of a binding pair is any binding partner which exists or can be generated for a first member/analyte. The first and the second member bind to each other at a single binding site. Preferably, the second member is the natural binding partner of the first member and is found complexed to the first member in vivo.

As defined herein, a “single binding site” means that only one molecule of first member will bind per molecule of second binding member.

As defined herein, a “sample” means any biological material suspected of containing an analyte/first member of interest.

As defined herein, “agglutination” means the formation of a complex which comprises at least two particles and manifests as a change in the turbidity of a sample compared to the turbidity of a sample in which the particles are not complexed.

A “reaction-enhancing polymer” means a generally hydrophilic polymer which prevents non-specific absorption to particles bound to first or second members and/or which increase the concentration of reactants in the solution by displacing water.

### DESCRIPTION OF PREFERRED EMBODIMENTS

The analyte detection methods according to the present invention rely on the detection of complexes formed between particles bound to a member of a binding pair, each member of the binding pair bindable to the other at one and only one binding site. In a preferred embodiment of the invention, the presence of the free form of a member of a binding pair is detected by detecting the agglutination of these particles. Only the presence of the free form will agglutinate the particles. Because bound or modified forms of a member of the binding pair will not agglutinate, the methods according to the invention provide highly specific assays to detect and/or quantitate the amount of a member of a binding pair which is free in a sample.

Analytes which can be assayed for according to the methods of the invention include any first member of a binding pair for which a binding partner exists or can be generated for, and can be naturally occurring in a biological sample, or artificially created. First members include, but are not limited to proteins and other types of binding members include antigens, peptides, amino acids, haptens (androgens, estrogens, progestogens, corticoids, thyroid hormones, digoxin, phenobarbital, phenytoin, and derivatives thereof), lipopolysaccharides, steroids, vitamins, drugs, nucleic acids, aptamers, and fragments, conjugates, and/or derivatives thereof.

The second member of a binding pair is any binding partner which exists or can be generated for a first member/analyte. For example, a second member corresponding to a first member which is a ligand can be a receptor, an antibody which binds to the ligand (including recombinant antibodies, antibody fragments, single-chain antibodies, and double-chain antibodies), an aptamer, and others. A second member corresponding to a first member which is a nucleic acid can be another nucleic acid, or a protein, polypeptide, or fragment of a protein or polypeptide, which can bind to the nucleic acid in either a sequence specific or non-sequence specific manner, an antibody, and others. It should be apparent to those of skill in the art that any given first member may have a plurality of second members. In a preferred embodiment, the second member is a molecule found bound to the first member in a biological sample. However, in certain embodiments, in may be desirable to generate a second member, using recombinant techniques, for example, to test the amount of free first member in a solution of first member and second member (for example, to measure the binding affinity of a first member to a recombinant second member). Artificially created binding pairs and first and second members are thus encompassed within the scope of the invention. Detection of the analytes/binding pairs may thus be performed in both biological (e.g., blood, plasma, serum, saliva, CSF, urine, culture media, cell suspensions) and non-biological samples (e.g., buffers and any other fluid suspected of containing an analyte/first member of interest).

Protein analytes contemplated within the scope of the invention comprise proteins which are found in both free and bound forms circulating in a biological fluid (e.g., blood) and include but are not limited to: protein S:C4BP; hormone binding proteins and their ligand hormones, such as insulin growth factor binding proteins (IGFBP-1, -2, -3, -4, -5, -6):insulin growth factor (IGF-I, IGF-II); growth hormone binding protein (GH-BP):growth hormone (GH); epidermal growth factor binding protein (EGF-BP):epidermal growth factor (EGF); platelet derived growth factor binding protein (PDGF-BP):platelet derived growth factor (PDGF); nerve growth factor binding protein (NGF-BP) and nerve growth factor(NGF); insulin binding protein (IBP):insulin; corticotropin releasing factor binding protein (CRF-BP):corticotropin releasing factor (CRF); transforming growth factor-β binding protein (TGF-β BP):transforming growth factor-(TGF-β); activin binding protein:activin (follistatin); binding pairs involved in the metabolism of lipids; and the like. It should be clear to those of ordinary skill in the art that the method may be used to quantitate the amount of either unbound member of a given binding pair and is generally useful to assay any member of a binding pair that is found in a biological sample.

The methods according to the invention are performed in two basic formats, a direct assay format and an inhibition assay format. In the direct assay format, the agglutination of particles is proportional to concentration of the free form of the first member of the binding pair (i.e., the analyte) in the sample. In the inhibition assay format, the free form of the member of the binding pair inhibits the agglutination reaction, and the extent of the inhibition is inversely proportional to the concentration of the free form of the member of the binding pair in solution. The methods according to the invention are simple to perform, low cost, highly sensitive and reproducible and therefore well-suited for use in clinical diagnostic tests. Reagents for performing the methods of the invention include compositions and kits to facilitate the convenience and reproducibility of the assay.

Direct Assay Format

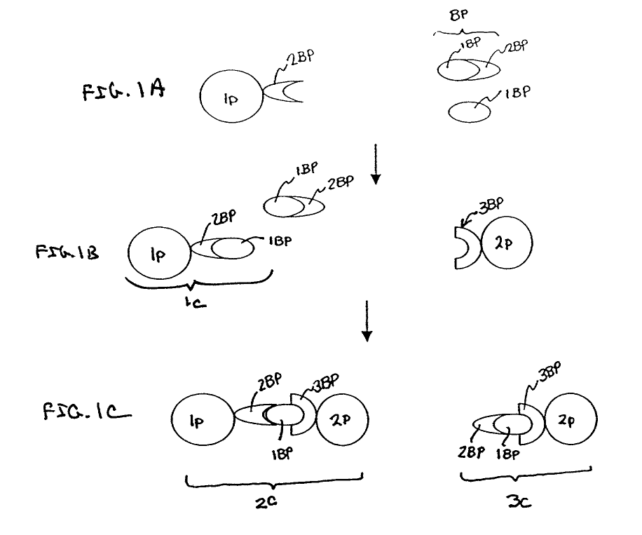

In one embodiment of the invention, shown in FIG. 1A, a method using a direct assay format is provided to detect the free form of the first member 1BP of a binding pair BP comprising a first 1BP and second member 2BP. In step 1 of the method, a first particle 1p bound to the second member 2BP is provided in a suitable reaction buffer at a concentration of between about 5×1010 and 1×1013 particles per ml. The particle solution is contacted with an aliquot of sample (e.g., human plasma or other body fluids or fluid samples), which is preferably about 1/20 the volume of particle solution forming a reacted sample.

Referring now to FIG. 1B, first member 1BP present in free form in the reacted sample solution will bind to the second member 2BP bound to the first particle 1p surface forming a first complex 1c. The amount of first member 1BP covering the particle 1p surface is proportional to the amount of free first member 1BP concentration in the sample. Because the first member 1BP has only a single binding site for the second member 2BP, no agglutination occurs at this stage.

Because the concentration of free and bound 1BP in solution is governed by the law of mass action, depletion of free 1BP will shift the chemical equilibrium in the sample, causing the 1BP:2BP complex to disassociate. However, it is the unexpected discovery of the present invention that an accurate determination of free 1BP can be made by minimizing the incubation of the reacted sample and optimizing the concentrations of components in the solution. Accordingly, the reacted sample is incubated for a sufficiently short period time to essentially avoid any shift in the chemical equilibrium in the sample which would cause the complexed form of the first member and the second member to disassociate, substantially preventing release of more free first member 1BP into the sample. Minimizing incubation times thus minimizes the possibility of overestimating the amount of the free form of the first member 1BP in the sample. According to a preferred embodiment of the invention, the reacted sample is incubated for 0 to about 180 seconds at a temperature which maintains the binding activity of the first 1BP and second member 2BP, e.g., preferably at about 37° C.

In the second step, as shown in FIG. 1B, a second particle solution comprising second particles 2p bound to a third member 3BP at a concentration of about 5×1011 and 1×1014 particles per ml is contacted with the reacted sample for a period sufficient to allow agglutination, preferably between about 2-5 minutes. The third member 3BP is capable of binding to the first member 1BP but is different from the second member 2BP; i.e., it binds at a different, single binding site on the first member. The second particle 2p itself may be the same as, or different from, the first particle 1p. The third member 3BP bound to the second particle 2p will bind to any first complex 1c, forming a second complex 2c illustrated in FIG. 1C. The third member 3BP will also bind to any remaining free first member 1BP in solution and to first member 1BP bound to second member 2BP (see complex 3c in FIG. 1). However, only formation of the second complex 2c and the formation of a bridge between first and second particle, 1p and 2p, will be detectable as an agglutination reaction, measurable as a change in turbidity of the sample.

The sensitivity and specificity of the assay can be enhanced by using reaction-enhancing polymers to stabilize particles 1p and 2p. In one embodiment, a polyhydroxylated polymer, such as polyethyleneglycol (PEG), is added to the particle solution. Other polymeric materials useful in the invention include, but are not limited to, polyvinylpirrolidone (PVP); dextran sulfate, proteins, such as, bovine serum albumin (BSA), nonfat dry milk, casein, or gelatin; and generally hydrophilic polymers. The polymers can be used to prevent non-specific absorption to the particles 1p and 2p, functioning like blocking agents, and/or can increase the concentration of reactants in the solution by displacing water. Polymers may be added at any time during the reaction, but preferably are added when the particle-binding member solutions are generated.

While absolute concentrations are not critical in practicing the invention, ratios of second member 2BP to third member 3BP in the reacted sample are optimized to minimize the disassociation of 1BP:2BP complex. According to a preferred embodiment of the invention, the molar ratio of 3BP to 2BP is between about 2 and 20. More preferably, the ratio is between about 5 and 10. The ratio of 3BP in the reaction buffer should also be higher than the amount of free first member 1BP in solution. In one embodiment of the invention, the ratio of 3BP is between about 5-100 times the amount of free first member 1BP in the sample. More preferably, the molar ratio of third member 3BP is between about 10 and 40 to times the amount of free first member 1BP in the sample. Reagents can be titrated using known samples of free first member 1BP to obtain maximal assay sensitivity and specificity; such titration steps are routine to those of skill in the art.

Step 1, including the first particle 1p bound to the second member 2BP, and step 2, including a second particle 2p bound to a third member 3BP, of the direct assay may be separated in time or occur simultaneously, providing both the first particle 1p bound to the second member 2BP and the second particle 2p bound to the third member 3BP at the same time. In one embodiment of the invention, first particle 1p bound to the second member 2BP and the second particle 2p bound to third member 3BP may be pre-mixed before adding these to the sample, since the second member 2BP and the third member 3BP do not significantly bind to each other. In a preferred embodiment of the invention, steps 1 and 2 are performed sequentially.

Agglutination, and thus the amount of free first member 1BP in the sample, can be quantitated by determining the amount of light scattering, or transmitted light intensity (non scattered light) in the reacted sample after step 2, measured in absorbance units. The absorbance units determined in a given experiment are correlated with absorbance units obtained for known concentrations of free first member 1BP through a calibration curve to determine the concentration of free first member 1BP in the sample.

Many optical instruments have been designed to measure particle agglutination by recording scattered or transmitted light intensity in a sample. The most popular of such devices are turbidimeters, or spectrophotometers, that measure the change in transmitted light intensity as a result of the agglutination process. In one embodiment of the invention, a coagulometer (e.g., ACL Futura, Instrumentation Laboratory®, 101 Hartwell Avenue, Lexington, Mass. 02421) is used to measure the turbidity of the sample. Coagulometers are widely used to quantify coagulation factors and related proteins in many hematology laboratories. Most coagulometers are able to measure the increase in turbidity of the reagent in the presence of a sample by recording the decrease of light transmitted or the increase of light scattered at a certain angle. In one embodiment according to this invention, agglutination of particles 1p and 2p is determined using a coagulometer essentially as a turbidimeter. In a preferred embodiment of the invention, the coagulometer is part of an automated system in which reagent handling is also automated to enhance the reliability and efficiency of the assay.

In an alternative embodiment, Flow Particle Analyzers (FPAs) are used to measure light scattered from single particles or particle aggregates rather than from particles in bulk solution. In this embodiment, light scattering by particles will depend on the refractive index of the particle, particle size, and particle concentration. It will be apparent to those of skill in the art that many instruments known in the art may be used to measure agglutination, and that use of all of these instruments is contemplated by this invention.

Inhibition Assay Format

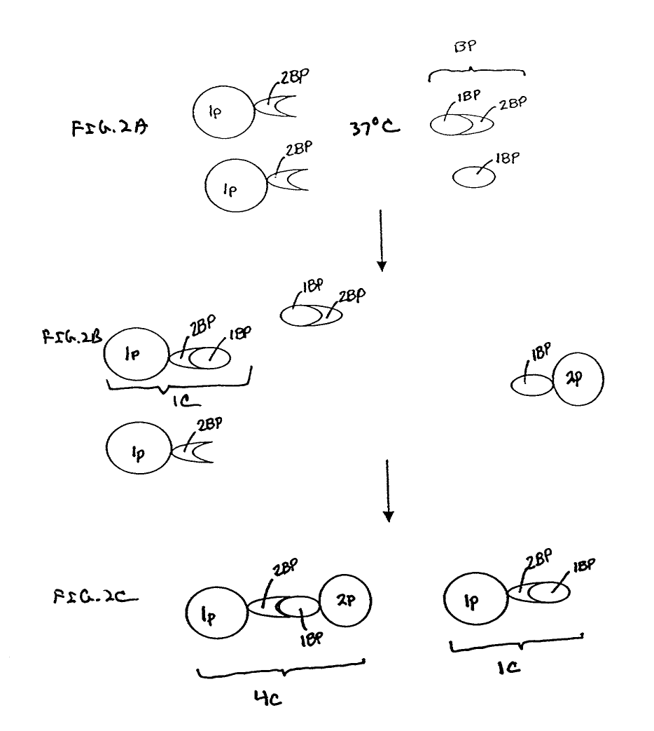

In another embodiment of the invention, shown in FIG. 2, an inhibition assay format is used to detect the free form of a first member 1BP which is a member of a binding pair BP, comprising 1BP and 2BP. In step 1 of this embodiment, referring now to FIG. 2A a first particle 1p bound to the second member 2BP is reacted with a sample of interest and the sample is incubated at a temperature which promotes binding, preferably 37° C. Any free form of 1BP will react with the second member 2BP bound to the first particle 1p to form a first complex 1c show in FIG. 2B. No agglutination occurs at this step.

In a second step, referring now to FIG. 2B a second particle 2p, which may be the same or different as the first particle 1p, bound to first member 1BP is added. The first member 1BP bound to the second particle 2p will react with any second member 2BP bound to the first particle 1p which has not yet reacted with free first member 1BP. The formation of a complex 4c, shown in FIG. 2C, comprising first particle 1p:second member 2BP:first member 1BP:second particle 2p, causes an agglutination reaction which is detectable. The amount of complex 4c, or agglutination, is inversely proportional to the amount of free first member 1BP present in the sample. As in the direct assay format, steps 1 and 2 can be performed either simultaneously or sequentially. However, unlike the direct assay format, the first particle 1p bound to the second member 2BP and second particle 2p bound to the first member 1BP cannot be pre-mixed. As in the direct assay format, reaction-enhancing polymers (e.g., PEG, PVP, BSA, nonfat milk, casein, gelatin, and others) may be added to increase the specificity and sensitivity of the reaction. Light scattering is measured using any of the devices disclosed above. However, in this assay format, increase in absorbance will be inversely proportional to the amount of free first member 1BP in the sample.

Compositions and Kits

In one embodiment, compositions are provided to supply standardized reagents for use in the assays disclosed above. Compositions for performing direct assays include first particles 1p bound to second member 2BP and second particles 2p bound to third member 3BP. The third member 3BP, while binding to the first member 1BP does so at a different single binding site from the second member 2BP. In a preferred embodiment, compositions are provided for use in direct assays to detect free protein S. In this embodiment, the composition includes first particles 1p bound to C4BP and second particles 2p which bind to an antibody which specifically binds to free protein S, but does so at a single binding site other than the C4BP binding site.

Compositions for use in an inhibition assay format include first particles 1p bound to second member 2BP and second particles 2p bound to first member 1BP. Compositions for use in an inhibition assay to detect free protein S will thus comprise first particles bound to C4BP and second particles bound to protein S.

In a further embodiment of the invention, the compositions according to the invention are provided pre-packaged in a kit, which may optionally comprise suitable buffers, reaction-enhancing polymers (e.g., PEG, PVP, BSA, nonfat milk, casein, gelatin, and others) and controls (e.g., known concentrations of free first member 1BP, such as protein S). In a preferred embodiment of the invention, kits according to the present invention provide standardized reagents for use in diagnostic assays to detect thrombophilia through the detection of free protein S in a sample. Kits may be provided with and without written instructions.

Particles according to the present invention are made of any material capable of being modified to present a binding molecule on some portion of its surface. Particles include, but are not limited to, synthetic polymeric material, colloidal metal particles, gelatin, glass, acrylamide, methacrylate, nylon, microscopic oxide powders, magnetic derivatives thereof, acrylonitrile, polybutadiene, dextrose, cellulose, polystyrene, polyvinyltoluene and/or derivatives thereof, and co-polymers, including, for example, styrene-acrylate, styrene-butadiene, styrene-divinylbenzene, and other latex polymers. Particles can comprise core polymers coated with other polymers, for example, poly(vinylbenzyl)chloride, and other equivalent substances. The type of polymer can be selected to optimize the specific gravity of a particle in a solution (e.g., sample fluid) (See, as disclosed in U.S. Pat. No. 5,585,278, for example, the entirety of which is incorporated herein by reference). In some embodiments of the invention, colored particles are provided and dispersed particles will have one color while aggregated particles will have another. In a preferred embodiment of the invention, the particle is inert to the sample.

The size of the particle is selected so as to be detectable by the eye or by another measuring instrument (e.g., a turbidometer or coagulometer) when aggregated. Light scattering intensity and distribution depends on two parameters. The first is the ratio of the refractive index of the particle to that of the sample fluid. For a given type of particle in solution, this ratio is fixed. The second parameter is the expressed by the ratio of πd/λ, where d is the particle diameter and λ is the wavelength of light in a vacuum. When d is much smaller than λ, the light scattered is very weak. As particle size increase, the amount of light which can be scattered by the particle increases.

Compositions for use in the invention preferably provide a ratio πd/λ of between about 0.2 and 3. At a wavelength of analysis of 405 nm, this means that particle size should optimally be between about 50 and 200 nm. In one embodiment of the invention, the size of the particles used ranges from about 50 nm to about 1000 nm, preferably between about 50 nm to about 500 nm, and most preferably, between about 100 nm to about 200 nm.

Particles can be uniform or variable in size. For example, in one embodiment of the invention, first and second particles are provided which are different sizes. In a preferred embodiment, the size of the first particle is between about 80 and 200 nm, and more preferably between about 100 and 150 nm. In this embodiment, a second particle is used which is smaller than the first particle. Particle size of the second reagent can range from about 50 nm and 150 nm, and preferably, between about 50 and 100 nm.

Particles according to the invention provide binding members (e.g., 2BP, 3BP) for binding to an analyte/free first member 1BP in a sample or for binding to other binding members bound to particles (e.g., 1BP bound to 2p, which binds to 2BP bound to 1p). Binding members can be provided on the surface of a particle using common techniques well know in the art, such as physical adsorption and chemical coupling.

Adsorption of molecules onto the hydrophobic surface of a particle is a rapid and spontaneous process. The driving force behind adsorption is the increase in entropy of a system and thus can be controlled by controlling the pH and ionic strength of a solution in which both the particle and molecule to be adsorbed onto the particle (e.g., the member of a binding pair) are provided. For example, protein adsorption may be maximized in a low ionic strength solution having a pH which is the same as the pI of the protein being adsorbed.

The amount of a member of a binding pair which can be absorbed onto a particle surface also depends on the surface area of the particle (e.g., m²/gm of particle). Because the surface area of a particle increases inversely its diameter, large particles will have smaller surface areas and decreased amounts of protein adsorbed per gm of particle. The amount of adsorption additionally depends on the “specific parking area” of the member and is affected by the molecular weight and tertiary structure of the member. For example, members which are globular proteins like immunoglobins have lower parking areas than fibrous proteins, like C4BP. The parking area of a protein can be determined by measuring the amount of bound protein to a particle surface under saturation conditions. By plotting bound protein against added protein isotherm curves can be generated which provide information about the plateau of protein saturation. The plateau can be optimized by controlling pH and ionic strength. Additional factors affecting protein adsorption include the isoelectric point and charge density of the protein. It should be apparent to those of skill in the art that a number of factors may be manipulated to increase the amount of binding member on the surface of a particle and that optimizing these factors requires only routine skill.

In another embodiment of the invention, a member of a binding pair is covalently coupled to a particle by exposing or creating an active group on the surface of the particle and reacting the active group with a reactive group on the member (which may be pre-existing or created). Examples of reactive groups include, but are not limited to, hydroxyl groups, carboxyl grounds, phenyl groups, thiol groups, amine groups, and linkers, for example, CH2Cl. In a preferred embodiment of the invention, a particle, for example, latex, is provided with pre-activated groups (for example, vinylbenzyl chloride, epoxide, aldehyde and others).

Considerations in attaching the member of the binding pair to the particle include: the type of bridge or linker between the member and the particle, and the need to preserve the accessibility of the binding site of the member being attached to the particle. Choice of conjugation chemistries and reactive groups are well known in the art and are disclosed in, for example, U.S. Pat. No. 5,858,648, U.S. Pat. No. 5,585,278, U.S. Pat. No. 5,095,097, U.S. Pat. No. 4,184,849, U.S. Pat. No. 4,210,723, U.S. Pat. No. 4,401,765, and U.S. Pat. No. 4,480,042, and Brinkley, “A Brief Survey of Methods for Preparing Protein Conjugates with Dyes, Haptens, and Cross-linking Reagents,” Bioconjugate Chemistry 3: 2-13, 1992, the entirety of which are incorporated herein by reference.

As discussed above, the binding member bound to the particle can be any binding partner which exists or can be generated for a first member/analyte. Binding members include, but are not limited to, antigens, receptors, ligands, haptens, antibodies (including recombinant antibodies, antibody fragments, single-chain antibodies, and double-chain antibodies), proteins (e.g., protein S), peptides, amino acids, hormones, steroids, vitamins, drugs, nucleic acids, combinations of nucleic acids and proteins, PNA molecules, and fragments, conjugates, and/or derivatives thereof. It should be recognized that for every analyte there can be a variety of suitable binding members.

Binding members can be obtained by isolating these molecules from a sample and can be natural, synthetic, or derived from a recombinant source. In one embodiment of the invention, C4BP is the binding member and is used generate 1p-2BP particles for use in either the direct assay format or the inhibition assay format. C4BP can be purified essentially free of protein S at a concentration of between about 0.2-5 mg/ml according to methods known in the art. See, e.g., Dahlbäck, Biochem J. 209: 847-856 (1983), the entirety of which is incorporated by reference herein.

Examples of third member 3BP molecules which can be used when C4BP is the second member 2BP (e.g., as in the direct assay format) include monoclonal antibodies which selectively react with protein S at a single site which is other than the single C4BP binding site. Methods of isolating antibodies are well known in the art and antibodies can be screened for by assaying for molecules that bind to protein S while not interfering substantially with C4BP binding. Antibodies can be recombinant, single-chain, double-chain, and include fragments which include the protein S-binding region of the antibody. Commercial antibodies can also be used. Preparation of an exemplary antibody, the HPS21 antibody, is described in Dahlbäck, et al., Journal of Biological Chemistry 265(14): 8127-8135 (1990), the entirety of which is incorporated by reference herein.

Protein S can also be purified essentially free of C4BP to generate 2p-1BP particles for use in an inhibition assay format by methods routinely used in the art. See, e.g., Dahlbäck, Biochem J. 209: 837-846 (1983). Isolated proteins can be stored at or below −40° C. until adsorption or attachment to particles.

### EXAMPLES

### A. Preparation of Reagents

i. Preparation of the C4BP-Latex Particles

In one embodiment of the invention, polystyrene latex particles (Estapor®) having a diameter of about 100 nm are sensitized by passive adsorption with purified C4BP at a ratio of 1.5-2.5 mg of C4BP per m2 of latex surface. Sensitization is performed by incubation in a borax buffered media at pH 8.2 (ionic strength between 0.05-0.2) at room temperature. Incubation times are optimized to maximize adsorption, and can range from about 2 to about 18 hours. In one embodiment, C4BP-latex particles are subsequently absorbed with bovine serum albumin under saturation conditions (e.g., at a ratio of 2 mg of BSA per mg of latex particles) to completely cover the surface of the particle. Incubation of C4BP-sensitized latex particles with BSA is performed by incubation at room temperature for an additional 2-18 hours.

C4BP-sensitized latex is then diluted in a reaction buffer containing 2 mM CaCl2 to obtain a final latex concentration of between about 4-8×1011 particles per ml. In one embodiment, a reaction-enhancing polymer such as PEG is added to the reaction buffer at a concentration between about 0.1-2.0% weight/volume. BSA can additionally be added to stabilized the particles at a concentration of between about 0.1-1.0% weight/volume in the final reagent. Reaction buffer comprising C4BP sensitized latex can be stored at preferably between 2-8° C., by itself, or mixed with monoclonal anti-protein S-latex particle reagent, discussed further below.

ii. Preparation of Monoclonal Anti-Protein S-Particles

Polystyrene latex particles (Estapor®) with a diameter of 70 nm are sensitized by passive adsorption with purified monoclonal anti-protein S (HPS-21) in a ratio of 2-2.5 mg of antibody per m² of latex surface. Sensitization is performed by incubation in a 2-Morpholino-ethanesulfonic acid (MES) buffered media at pH 6.0 (ionic strength of 0.005-0.020) for between about 2-18 hours. As above, complete saturation of the latex surface is accomplished by incubating the anti-protein S-latex particles with BSA under saturation conditions, e.g., providing a ratio of 2 mg of BSA per mg of latex particles and incubating the anti-protein S-sensitized latex particles with BSA at room temperature for between about 2-18 hours.

Anti-protein S sensitized latex particles are then diluted in reaction buffer (Borax pH 8.2; ionic strength between 0.05-0.2) containing 2 mM CaCl2 to achieve a final particle concentration of between about 2-8×1012 particles per ml. As above, BSA and PEG can be added as reaction-enhancing polymers at a concentration in a range between about 0.1-2.0% weight/volume

### B. Reaction Conditions

In one embodiment, 12 μl of non-diluted plasma sample is introduced into the reaction cell of an automated coagulation analyzer (ACL Futura, from Instrumentation Laboratory®). After a short incubation to reach a working temperature of 37° C. (e.g., 30-150 seconds), C4BP-latex particles are added at a concentration of 6×10¹¹ particles/ml in a volume of 150 μl and mixed with the plasma sample. The plasma sample/C4BP-latex particle mix is incubated for 0-180 seconds after which time 80 μl of monoclonal anti-protein S-latex particles is added to the reaction cuvette. The particles are allowed to agglutinate (forming complexes of particle:C4BP:protein S:anti-protein S:particle) for 2-5 minutes.

While absolute concentrations are not critical in practicing the invention, ratios of C4BP- to anti-protein S in the reacted sample should be optimized. According to a preferred embodiment of the invention, the molar ratio of anti-protein S to C4BP is between about 2 and 20. More preferably, the ratio is between about 5 and 10. The amount of anti-protein S in the reaction buffer should also be higher than the amount of free protein S in solution. In one embodiment of the invention, the ratio of anti-protein S is between about 5-100 times the amount of free protein S in the sample. More preferably, the molar ratio of anti-protein S is between about 10 and 40 times the amount of free protein S in the sample. Reagents can be titrated using known samples of free protein S to obtain maximal assay sensitivity and specificity; such titration steps are routine to those of skill in the art.

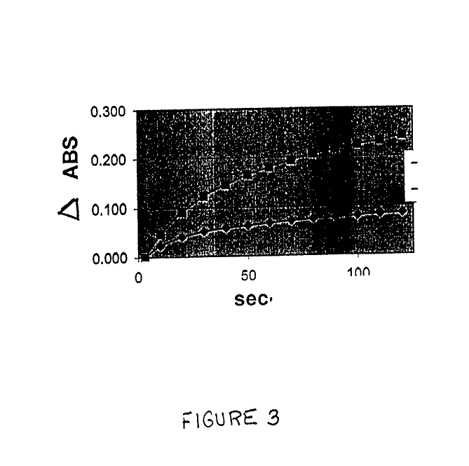

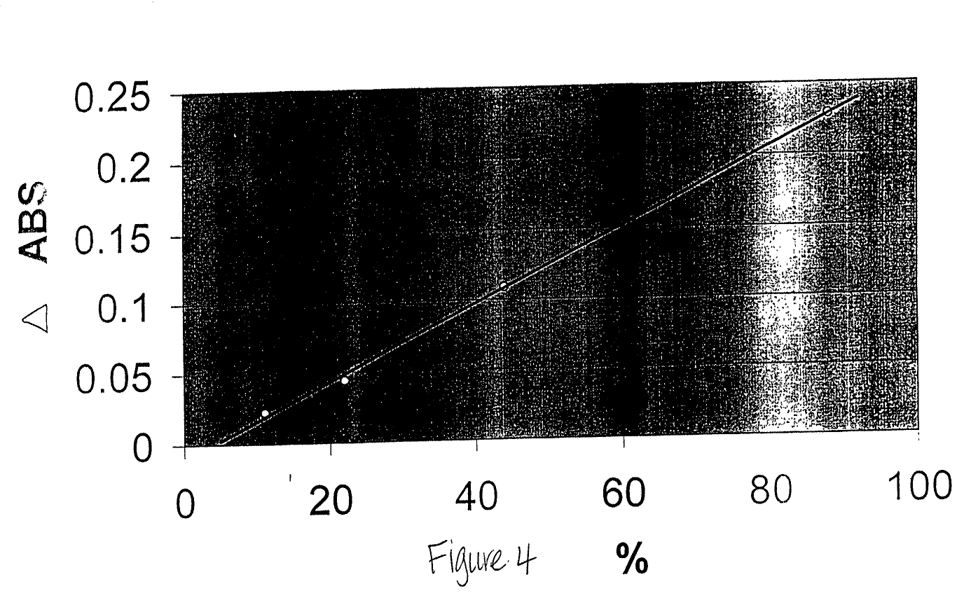

Agglutination is reflected by an increase in absorbance recorded after the initial mixing step and until the final incubation period. FIG. 3 shows a graph of free protein S (PS) over time measured as an increase in the absorbance measured at 405 nM in an ACL Futura Coagulometer in a sample from a healthy patient and in a sample from a patient with thrombophilia. The increase in absorbance can be correlated with amount of free protein S in the sample through a calibration curve derived by performing reactions with known concentrations of free protein S (preferably between 0.8-1.2 IU/ml) in a normal human plasma sample as shown in FIG. 4 (100% free protein S corresponds to 1.0 IU/ml). This provides a good correlation with the international standard prepared by a WHO international laboratory, the National Institute for Biological Standards and Control (NIBSC).

The instant invention thus provides a useful diagnostic assay for the detection of thrombophilia. As can be scene from Table I below, samples from patients diagnosed as having either Type I (lower concentrations of free protein S and total protein S) or Type III (lower concentrations of free protein S and normal concentrations of total protein S) thrombophilia have significantly decreased levels of free protein S as measured by this assay.

TABLE I
Determination of Free Protein S in Plasma

| | | Change in | Free Protein S |
|---|---|---|---|
| Sample No. | Patient Status | Absorbance | (% normality) |
| N201 | Normal | 0.2 | 84.62 |
| N202 | Normal | 0.219 | 84.46 |
| N203 | Normal | 0.201 | 80.32 |
| N204 | Normal | 0.252 | 99.32 |
| N205 | Normal | 0.192 | 76.85 |
| N206 | Normal | 0.172 | 69.29 |
| N207 | Normal | 0.294 | 115.0 |
| N208 | Normal | 0.231 | 91.44 |
| N209 | Normal | 0.307 | 120.0 |
| N210 | Normal | 0.316 | 123.1 |
| D101 | Type III | 0.108 | 43.65 |
| D102 | Type III | 0.083 | 34.57 |
| D104 | Type III | 0.073 | 30.81 |
| D105 | Type III | 0.056 | 24.58 |
| D106 | Type III | 0.069 | 29.41 |
| D110 | Type I | 0.084 | 34.83 |
| D113 | Type I | 0.093 | 38.10 |
| D114 | Type I | 0.034 | 19.94 |
| D123 | Type I | 0.088 | 36.81 |
| D124 | Type I | 0.026 | 14.47 |

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

What is claimed is.

1. A method for detecting an unbound form of a first member of a binding pair, the binding pair comprising a first and second member, each member bindable to the other, the method comprising the steps of:
(a) providing a first particle bound to the second member;
(b) reacting the first particle bound to the second member with a sample, thereby forming a first complex between the second member bound to the first particle and unbound first member present in said sample;
(c) providing a second particle bound to a third member, the third member being different from the second member and being capable of binding to the first member;
(d) reacting the second particle bound to the third member to the sample, thereby forming a second complex between the third member bound to the second particle and the first complex; and
(e) detecting any second complex formed.

2. The method of claim 1, wherein the third member is an antibody which specifically binds to the first member.

3. The method of claim 1, wherein the first and/or second particle is latex.

4. The method of claim 1, wherein the second complex is detected by measuring an increase in the turbidity of the sample.

5. The method of claim 1, wherein steps (a) through (d) are performed sequentially.

6. The method of claim 1, wherein steps (a) through (d) are performed simultaneously.

7. The method of claim 1, wherein the amount of second complex formed is quantitated.

8. The method of claim 1, wherein the first member is protein S.

9. The method of claim 1, wherein the second member is C4b-binding protein (C4BP).

10. The method of claim 1, wherein the sample is selected from the group consisting of blood, plasma, serum, saliva, CSF, urine, culture media, a cell suspension, a buffer and an artificially prepared fluid containing the first member.

11. The method of claim 1, wherein the second member binds to the first member at a single binding site.

12. The method of claim 11, wherein the third member binds to the first member at a single binding site which is different from the single binding site to which the second member binds.

13. The method of claim 1, wherein step (b) is performed within 0 to about 180 seconds.

14. The method of claim 1, wherein the molar ratio of third member to second member is between about 2 and 20.

15. The method of claim 1, wherein the molar ratio of the third member to second member is between about 5 and 10.

16. The method of claim 1, wherein the amount of third member is higher than the amount of free first member is the sample.

17. The method of claim 1, wherein the molar ratio of third member is between about 10 and 40 times the amount of free first member in the sample.

18. A composition for detecting an unbound form of a first member of a binding pair, the binding pair comprising a first and second member, each member bindable to the other, the composition comprising:
a first particle bound to the second member;
a second particle bound to a third member, the third member being different from the second member and capable of binding to the first member at a binding site different from the second member.

19. The composition of claim 18, wherein the first member is protein S and the second member is C4BP.

20. The composition of claim 18, wherein the third member is an antibody and the second member is not an antibody.

21. The composition of claim 18, wherein the second member comprises a single binding site for the first member.

22. The composition of claim 21, wherein the third member binds to the first member at a single binding site which is different from the single binding site to which the second member binds.

23. A method for detecting an unbound form of a first member of a binding pair, the binding pair comprising a first and second member, each member bindable to the other, the method comprising the steps of:
(a) providing a first particle bound to the second member;
(b) reacting the first particle bound to the second member with a sample, thereby forming a first complex between the second member bound to the first particle and unbound first member present in said sample;
(c) providing a second particle bound to the first member;
(d) reacting the second particle bound to the first member with the sample, thereby forming a second complex between second particle bound to the first member and first particle bound to second member which is not already bound to the first member; and
(e) detecting any second complex formed, wherein the amount of second complex formed is inversely proportional to the amount of unbound first member is the sample.

24. The method of claim 23, wherein the first and/or second particle is latex.

25. The method of claim 23, wherein the second complex is detected by measuring an increase in the turbidity of the sample.

26. The method of claim 23, wherein the amount of second complex formed is quantitated.

27. The method of claim 23, wherein the first member is protein S.

28. The method of claim 23, wherein the second member is C4BP.

29. The method of claim 23 wherein the sample is selected from the group consisting of blood, plasma, serum, or an artificially prepared buffer containing the first member.

30. A composition for detecting an unbound form of a first member of a binding pair comprising a first and second member, each member bindable to the other, the composition comprising:
a first particle bound to the second member; and
a second particle bound to the first member.

31. The composition of claim 30, wherein the first member is protein S and the second member is C4BP.

32. A method for diagnosing thrombophilia comprising performing the method of claim 8, and further comprising comparing the amount of second complex formed to the amount of second complex formed in a sample derived from an individual without thrombophilia.

33. A method for diagnosing thrombophilia comprising performing the method of claim 27, and further comprising comparing the amount of second complex formed to the amount of second complex formed in a sample derived from an individual without thrombophilia.
